(19) United States
(12) Patent Application Publication
(10) Pub. No.: US 2012/0237000 A1
(43) Pub. Date: Sep. 20, 2012

(54) COVERLET

(75) Inventor: Salwa Rahme, Redfern (AU)

(73) Assignee: Alara Group Pty. Ltd., Mortlake (AU)

(21) Appl. No.: 13/496,999

(22) PCT Filed: Sep. 15, 2010
(86) PCT No.: PCT/AU10/01194

§ 371 Date: Jun. 6, 2012

(30) Foreign Application Priority Data
Sep. 17, 2009 (AU) 2009904510

(51) Int. Cl. A61B 6/10

(52) U.S. Cl. 378/204

(57) ABSTRACT

A coverlet (10) for mounting on a CT scanner (12) or other medical equipment. The scanner (12) has a tunnel (14) with a front open end (16). The coverlet (10) includes a sheet (22) with a layer of fluid absorbing material (24) attached to a layer of fluid impervious material (26). The sheet (22) is adapted to cover at least part of the tunnel (14).

### TECHNICAL FIELD

This invention relates to a coverlet suitable for mounting on medical devices, particularly but not restricted to, the interior of a CT scanner.

### BACKGROUND OF THE INVENTION

It is generally desirable in the medical equipment industry to prevent cross contamination of pathogens between patients during medical procedures as a result of direct physical contact with the medical equipment.

The specification of U.S. Pat. No. 7,465,089 discloses a covering for preventing fluid ingress into a CT scanner and tube. This covering however has a shortcoming that it does not prevent or reduce infection of patients and/or instruments.

It is an object of the present invention to provide a coverlet which may overcome or ameliorate the above shortcoming or which will at least provide a useful alternative.

### DISCLOSURE OF THE INVENTION

It is intended that a coverlet of the present invention, at least in one embodiment, provides a sterile field for X-ray or other scanning procedures to be carried out.

According to one aspect of the present invention, there is provided a coverlet for mounting on a CT scanner or other medical equipment having a tunnel with a front open end, the coverlet including a sheet with a layer of fluid absorbing material attached to a layer of fluid impervious material, the sheet being adapted to cover at least part of the tunnel.

In a preferred embodiment, the tunnel for use with the coverlet of the invention is defined by a cylindrical hollow provided in a CT scanner. The sheet is preferred to be made of soft and pliable material capable of conforming to a curved surface of the tunnel.

Preferably, the layer of fluid absorbing material is backed by the layer of fluid impervious material. In use, the layer of fluid absorbing material is adapted to be in contact with a patient's body whilst the layer of fluid impervious material is in contact with the curved surface.

In this embodiment, if the tunnel has a rear open end, the sheet is preferred to extend from the front open end to the rear open end and includes a transparent section. Preferably, the transparent section is in register with a transparent band (provided in the CT scanner) through which X-rays can be transmitted and detected.

In a preferred embodiment, the coverlet includes a first extension which extends beyond the front open end of the tunnel. The first extension is preferred to include an apron which drapes over the front open end of the tunnel. Preferably, the apron includes a collar adapted to cover an exterior of a lower portion of the front open end of the tunnel. The apron may include a pocket adapted to receive waste material.

The sheet may include a second extension which extends beyond and drapes over the rear open end of the tunnel. The second extension is preferred to be configured such that it facilitates alignment of the sheet with the tunnel.

Optionally, the sheet is adhered to the curved surface (if present) by way of an adhesive element. The adhesive element may include adhesive tapes and/or Velcro™.

### BRIEF DESCRIPTION OF THE DRAWINGS

The invention may be better understood from the following non-limiting description of preferred embodiments, in which:

FIG. 1 is a perspective view of a coverlet in accordance with one embodiment of the present invention being mounted on a CT scanner;

FIG. 2 is a cross sectional view of a section of the coverlet of FIG. 1;

FIG. 3 is a cross sectional view of the CT scanner with the coverlet of FIG. 1 mounted thereon; and

FIG. 4 is a perspective view of a coverlet in accordance with another embodiment of the present invention being mounted on a CT scanner.

### DETAILED DESCRIPTION OF THE DRAWINGS

It should be noted that although a coverlet of the present invention is intended to be applied to a CT scanner, it may be customised so as to be applicable to other medical equipment or machinery.

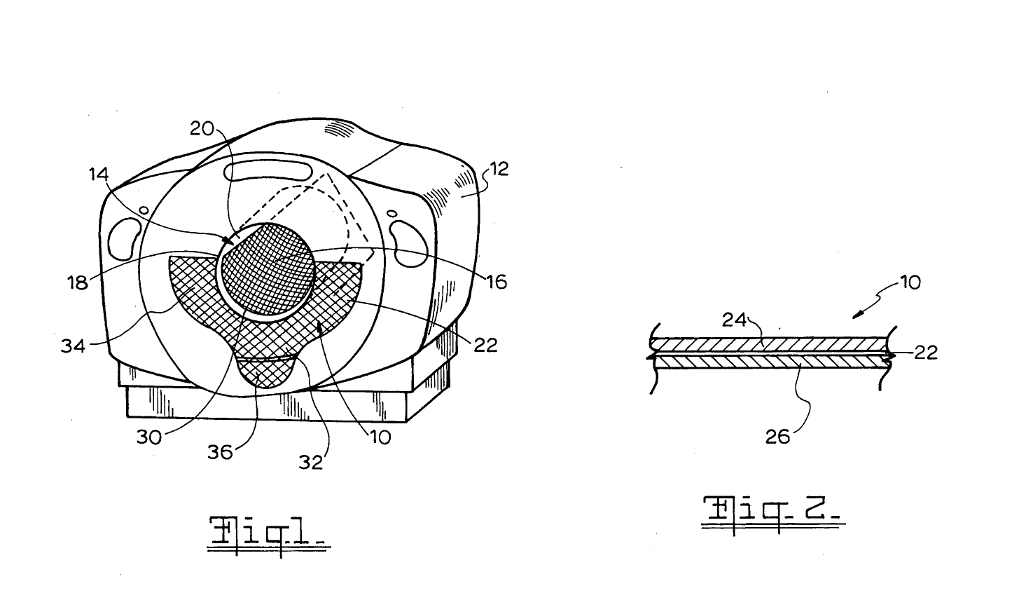

Referring to FIG. 1, a coverlet 10 is designed for mounting on a CT scanner 12 having a cylindrical hollow 20 defining a tunnel 14 with a front open end 16. The tunnel 14 has a curved surface 18.

Turning to FIG. 2, the coverlet 10 is in the form of a sheet 22 with a layer of fluid absorbing material 24 backed by a layer of fluid impervious material 26. The fluid absorbing material 24 and the fluid impervious material 26 may be bonded together by stitching or an adhesive. The sheet 24 is soft and pliable being capable of conforming to the curved surface 18 of the cylindrical interior 20 when it is placed thereon.

As shown in FIGS. 1 and 3, the sheet 22 covers a lower half of the tunnel 14. Once the sheet 22 is spread and placed on the curved surface 18, the layer of fluid absorbing material 24 faces upward such that in use it is in contact with a patient's body whilst the layer of fluid impervious material 26 is in contact with the curved surface 18.

Referring to FIG. 3, the tunnel 14 has a rear open end 28. The sheet 22 extends from the front open end 16 to the rear open end 28 with a transparent section 30. In the present embodiment, the transparent section 30 is located such that it is in register with a transparent band provided in the CT scanner through which X-rays can be emitted and detected. The transparent section 30 however may be provided in the proximity of the open end 28 instead, as shown in FIG. 1. Also, the sheet may include more than one transparent section in the event that a plurality of X-ray or other scans is required to take place simultaneously.

As best shown in FIG. 3, the coverlet 10 includes a first extension which extends beyond the front open end 16 of the tunnel 14. The first extension of the sheet 22 includes an apron 32 which drapes over the front open end 16 of the tunnel 14. The apron 32 includes a collar 34 (refer to FIG. 1) which covers an exterior of a lower portion of the front open end 16 of the tunnel 14. The apron 32 includes a pocket 36 configured to receive waste material.

The sheet 22 also includes a second extension in the form of a flap 38 which extends beyond and drapes over the rear open end 28 of the tunnel 14. The flap 38 is configured such that it facilitates alignment of the main part of the sheet 22 with the tunnel 14.

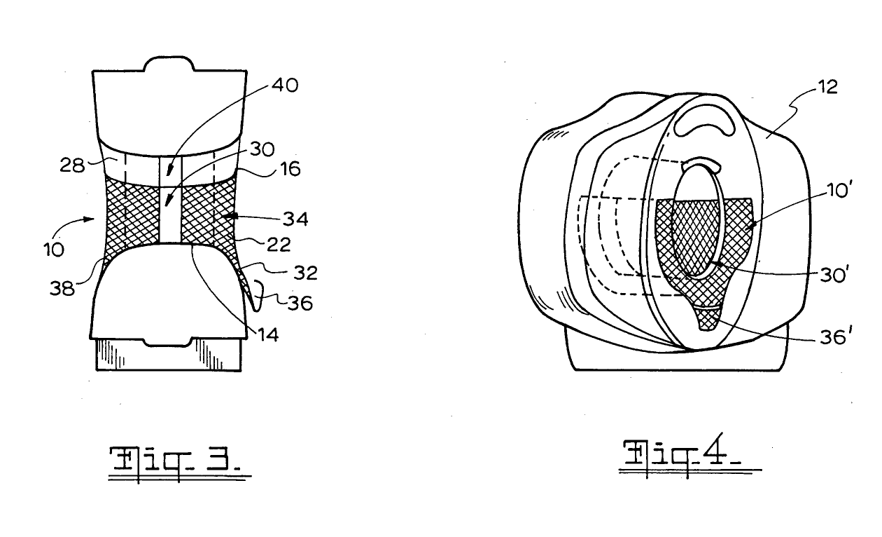

Referring now to FIG. 4, another embodiment of the coverlet 10′ of the present invention is shown without a flap extending beyond the rear open end 28. Also, this embodiment includes an additional transparent section 30′ located near the front open end 16.

Although not shown in the drawings, to maximise its stability and performance, the sheet 22 is adhered to the curved surface 18 by way of one or more adhesive elements such as adhesive tapes or hook and loop closures, such as sold as Velcro™.

Now that preferred embodiments of the present invention have been described in some detail, it will be apparent to a person skilled in the art that the coverlet may offer at least the following advantages:
1. it is easy to install and use;
2. it continuously covers at least the lower half of the interior of the tunnel of the CT scanner without requiring additional components; and
3. it can easily be retrofitted or replaced for each new patient to provide a sterile field inside the CT scanner thereby reducing the likelihood of cross contamination of pathogens between patients.

Those skilled in the art will appreciate that the invention described herein is susceptible to variations and modifications other than those specifically described. All such variations and modifications are to be considered within the scope and spirit of the present invention the nature of which is to be determined from the foregoing description.

### INDUSTRIAL APPLICABILITY

The coverlet of the present invention is industrial applicable in that it continuously covers at least the lower half of the interior of the tunnel of the CT scanner to provide a sterile field for X-ray or other scanning procedures to be carried out without requiring additional components, and can easily be retrofitted or replaced for each new patient thereby reducing the likelihood of cross contamination of pathogens between patients.

1-15. (canceled)

16. A coverlet for mounting on a CT scanner or other medical equipment having a tunnel with a front open end, the coverlet comprising a sheet with a layer of fluid absorbing material attached to a layer of fluid impervious material, the sheet being configured to cover at least part of the tunnel.

17. The coverlet of claim 16, wherein the sheet is made of soft and pliable material configured to conform to a curved surface of the tunnel, the curved surface being defined by a cylindrical hollow provided in the CT scanner or other medical equipment.

18. The coverlet of claim 16, wherein the layer of fluid absorbing material is backed by the layer of fluid impervious material.

19. The coverlet of claim 16, wherein when in use the layer of fluid absorbing material is configured to be in contact with a patient's body whilst the layer of fluid impervious material is in contact with the curved surface.

20. The coverlet of claim 16, wherein:
the tunnel has a rear open end;
the sheet is configured to extend from the front open end to the rear open end; and
the sheet further comprises a transparent section.

21. The coverlet of claim 20, wherein the transparent section is configured to be in register with a transparent band (provided in the CT scanner or other medical equipment) through which X-rays are capable of being transmitted and detected.

22. The coverlet of claim 16, wherein the coverlet includes a first extension configured to extend beyond the front open end of the tunnel.

23. The coverlet of claim 22, wherein the first extension includes an apron which is configured to drape over the front open end of the tunnel.

24. The coverlet of claim 23, wherein the apron includes a collar configured to cover an exterior of a lower portion of the front open end of the tunnel.

25. The coverlet of claim 23, wherein the apron includes a pocket configured to receive waste material.

26. The coverlet of claim 20, wherein the sheet includes a second extension configured to extend beyond and drape over the rear open end of the tunnel.

27. The coverlet of claim 26, wherein the second extension is configured such that it facilitates alignment of the sheet with the tunnel.

28. The coverlet of claim 17, wherein the sheet is configured to adhere to the curved surface by way of an adhesive element.

29. The coverlet of claim 28, wherein the adhesive element includes at least one of an adhesive tape and a hook and loop closure.
